(19) United States
(12) Patent Application Publication
(10) Pub. No.: US 2025/0304543 A1
(43) Pub. Date: Oct. 2, 2025

(54) PROCESS FOR MAKING A SUBSTANTIALLY NITROSAMINE-FREE, SUBSTANTIALLY PEROXIDE-FREE, SUBSTANTIALLY COLORLESS AMINE OXIDE

(71) Applicant: BASF SE, Ludwigshafen am Rhein (DE)

(72) Inventors: Peter OECHSLE, Ludwigshafen am Rhein (DE); Andreas KUNST, Ludwigshafen am Rhein (DE); Christian MUELLER, Ludwigshafen am Rhein (DE); Joaquim Henrique TELES, Ludwigshafen am Rhein (DE)

(21) Appl. No.: 18/872,854

(22) PCT Filed: Jun. 9, 2023
(86) PCT No.: PCT/EP2023/065472

§ 371 Date: Dec. 9, 2024

(30) Foreign Application Priority Data
Jun. 10, 2022 (EP) 22178338.4

(51) Int. Cl. C07D 295/24

(52) U.S. Cl. CPC C07D 295/24

(57) ABSTRACT

A process for making a substantially nitrosamine-free, substantially peroxide-free, substantially colorless amine oxide solution comprises reacting a tertiary amine capable of forming an amine oxide with a less than stoichiometric amount of aqueous hydrogen peroxide in an aqueous medium while imposing on the aqueous medium a vapor space having a carbon dioxide partial pressure p(CO₂) of less than 0.75 bar absolute. The process allows for obtaining amine oxides which are substantially nitrosamine-free, substantially peroxide-free, and substantially colorless. This unique combination of desirable features has not been achieved so far in a process only comprising oxidation and distillation.

The present invention relates to a process for making a substantially nitrosamine-free, substantially peroxide-free, substantially colorless amine oxide.

Amine oxides are commonly used as oxidizing agents, solvents or in consumer products such as shampoos, conditioners, detergents, and hard surface cleaners. An industrially important amine oxide representative is N-methylmorpholine N-oxide. Aqueous solutions of N-methylmorpholine N-oxide are technically used as a solubilizer in Lyocell fiber production, wherein pulp is dissolved and spun from a solution in N-methylmorpholine N-oxide and water.

Generally, amine oxides are produced by oxidation of tertiary amines, e.g. by using hydrogen peroxide as an oxidant. The processes known from the art commonly use excess oxidant, e.g. hydrogen peroxide, and are thus often accompanied by disadvantages such as contamination of the amine oxide product with unreacted oxidant, e.g. hydrogen peroxide.

Nitrosamines are formed as minor by-products in the preparation of amine oxides using aqueous hydrogen peroxide. Although the amount of nitrosamine is very small, this small amount renders the amine oxide unsuitable in many applications that involve human contact. This is because nitrosamines are reported to be carcinogenic and/or mutagenic.

B. Balagam et al., Inorg. Chem. 2008, 47, 1173-1178 relates to hydrogen peroxide N-oxidation of amines and describes the oxidation of N-methylmorpholine using CO₂ and H₂O₂ in a round bottomed flask.

T. Baumeister et al., React. Chem. Eng. 2019, 4, 1270-1276 relates to the continuous flow synthesis of amine oxides by oxidation of tertiary amines and describes the oxidation of N-methylmorpholine using H₂O₂ under flow conditions in a microstructured reactor.

The EP 0 307 184 describes a process for oxidizing a tertiary amine with hydrogen peroxide to form a tertiary amine oxide. For this purpose, the tertiary amine is contacted with aqueous hydrogen peroxide in the presence of a promoter formed by adding carbon dioxide to the reaction mixture. The reaction is conducted at a temperature below about 45° C., whereby the nitrosamine content of the tertiary amine oxide product is substantially reduced compared to the nitrosamine content of tertiary amine oxides produced by the same process at higher temperatures.

The U.S. Pat. No. 5,693,796 describes a process for reducing the nitrosamine content of N-methylmorpholine N-oxide. A catalytic amount of a carboxylic acid selected from formic acid, acetic acid, propionic acid, butyric acid and benzoic acid or a carboxylate salt thereof is added to an aqueous solution of N-methylmorpholine and oxidation is carried out using hydrogen peroxide.

The EP 0 320 694 describes a process for making a substantially nitrosamine-free amine oxide, comprising reacting a tertiary amine capable of forming an amine oxide with aqueous hydrogen peroxide in the presence of the synergistic combination of ascorbic acid and a promoter formed by adding carbon dioxide to the reaction mixture. Excess amounts of hydrogen peroxide are preferred; such excess H₂O₂ remaining after the reaction must be destroyed by the addition of a reducing agent or a peroxide decomposition catalyst such as manganese dioxide.

The U.S. Pat. No. 5,216,154 describes the preparation of N-methylmorpholine N-oxide by contacting N-methylmorpholine with a molar excess of hydrogen peroxide in an atmosphere consisting essentially of carbon dioxide. This process uses carbon dioxide as a promoter to form N-methylmorpholine N-oxide containing very low amounts of nitrosamines.

The U.S. Pat. No. 6,166,255 describes the oxidation of tertiary amines with aqueous hydrogen peroxide in an aqueous reaction medium at about 15 to 25° C. using carbon dioxide as a catalyst. While agitating the reaction mixture, the temperature is allowed to rise adiabatically to about 50 to 100° C. In this way, it is possible to produce tertiary amine oxides with very low levels, if any, of nitrosamine impurity, e.g. below 30 ppb, without addition of metal and/or phosphorus-containing components.

By using carbon dioxide as a promoter as described in the references above, the issues about undesirable contents of nitrosamine have been eliminated, or at least mitigated them to an acceptable level. Besides nitrosamine contamination, however, there is the problem that colored species are formed as reaction by-products which negatively impact product quality, even in low concentrations. The thus obtained deeply yellow-brown solutions of amine oxide are not suitable for all applications unless further purification steps are undertaken.

The U.S. Pat. No. 5,502,188 describes a process for producing aqueous solutions of N-methylmorpholine N-oxide having APHA numbers below 200, based on a N-methylmorpholine N-oxide content of 50% by weight. For this purpose, an aqueous N-methylmorpholine solution having a water content of at least 35% by weight is oxidized with hydrogen peroxide. After the reaction, the concentration of the aqueous N-methylmorpholine N-oxide solution is adjusted by distilling off residual N-methylmorpholine and water. However, there remains a need for further reduction of colored species.

Therefore, it is an object of the present invention to provide an improved process for preparing amine oxides which give rise to substantially nitrosamine-free, substantially peroxide-free, and substantially colorless amine oxides.

This object has been solved by a process for making a substantially nitrosamine-free, substantially peroxide-free, substantially colorless amine oxide solution. Said process comprises reacting a tertiary amine capable of forming an amine oxide with a less than stoichiometric amount of aqueous hydrogen peroxide in an aqueous medium while imposing on the aqueous medium a vapor space having a carbon dioxide partial pressure p(CO₂) of less than 0.75 bar absolute, preferably less than 0.20 bar absolute.

Although carbon dioxide is an effective promoter for the conversion of a tertiary amine into its amine oxide, applicants have found that amounts of free carbon dioxide in excess of a promoting amount can lead to undesired discoloration. By the process of the invention, it is possible that the amount of carbon dioxide in the aqueous medium, in whatever form it exists, is not higher than a promoting amount of carbon dioxide. Without wishing to be bound by theory, we believe that a promoting amount of carbon dioxide is present as peroxycarbonate or other adduct of hydrogen peroxide and carbonate. “Free” carbon dioxide species, i.e. excess carbon dioxide species that are not converted to peroxycarbonate by the available hydrogen peroxide, are believed to be responsible or at least contribute to the abovementioned discoloration problems.

The process of the invention comprises reacting a tertiary amine capable of forming an amine oxide with aqueous hydrogen peroxide in an aqueous medium. Generally, an aqueous solution of the tertiary amine is placed in a reaction vessel and aqueous hydrogen peroxide is metered-in over a variable timespan.

The process of the invention is applicable to any tertiary amine capable of forming an amine oxide. These are well known to the skilled person. They include amines which do not have a hydrogen atom bonded to the amine nitrogen atom. Such amines include trialkylamines; triarylamines; triarylalkylamines; mixed alkyl-aryl, alkyl-arylalkyl, aryl-arylalkyl or alkyl-aryl-arylalkylamines; tricycloalkylamines; alkyl-cycloalkylamines; aryl-cycloalkylamines; cyclic amines; for example: butyl dimethylamine, hexyl dimethylamine, isobutyl dimethylamine, 2-ethylhexyl dimethylamine, octyl dimethylamine, decyl dimethylamine, dodecyl dimethylamine, tetradecyl dimethylamine, hexadecyl dimethylamine, eicosyl dimethylamine, docosyl dimethylamine, triacontyl dimethylamine, tributylamine, butyl diethylamine, isobutyl diethylamine, decyl butyl ethylamine, hexadecyl hexyl methylamine, eicosyl dibutylamine, trioctylamine, tridodecylamine, dieicosyl ethylamine, ditriacontyl methylamine, N,N,-dimethylaniline, N-methyl-N-dodecylaniline, cyclopentyl dimethylamine, cyclohexyl dimethylamine, dicyclohexyl methylamine, cyclododecyl dimethylamine, diphenyl butylamine, p-tolyl diethylamine, α-naphthylbutylmethylamine, benzyl butyl methylamine, α-methylbenzyl butyl methyl-amine, 4-butylbenzyl octyl methylamine, dibenzyl butylamine, 4-pentyl-benzyl dibutyl-amine, N-butylmorpholine, N-methylmorpholine, N-methylpiperidine, N-dodecyl-piperidine, N-octadecylpiperidine, N-triacontylpiperidine, N-methylpiperazine, N-butyl-piperazine, N-octylpiperazine, N-phenylpiperidine, N-benzylpiperidine, N-cyclohexyl-piperidine, pyridine and the like. In view of the high industrial demand of N-methylmorpholine N-oxide, an important aspect of the invention is an embodiment where the tertiary amine is N-methylmorpholine.

In an embodiment, the initial concentration of tertiary amine in the aqueous medium, that is the concentration of tertiary amine prior to the addition of hydrogen peroxide, is in the range of from 40 to 95 vol.-%, preferably 60 to 85 vol.-%.

Any aqueous hydrogen peroxide can be used. In view of practical considerations, the concentration of hydrogen peroxide in the aqueous hydrogen peroxide that is added to the aqueous medium is in the range of from 10 to 70 wt.-%, preferably 29 to 51 wt.-%.

According to the invention, a carbon dioxide partial pressure p(CO₂) of less than 0.75 bar absolute is imposed on the aqueous medium. “Imposing a carbon dioxide partial pressure p(CO₂) on the aqueous medium” is intended to mean that in the vapor space above the aqueous medium and in direct contact with the aqueous medium, a defined carbon dioxide partial pressure is maintained.

The prior art, to the best of our knowledge, exclusively discloses processes with a carbon dioxide partial pressure of 1 bar absolute. By the carbon dioxide partial pressure p(CO₂) of less than 0.75 bar absolute of the invention, the amount of carbon dioxide which dissolves in the aqueous medium may be controlled: There is an equilibrium between the dissolved carbon dioxide and the carbon dioxide in the gas phase. High carbon dioxide partial pressures result in high amounts of carbon dioxide being dissolved in the aqueous medium, whereas low carbon dioxide partial pressures result in low amounts of carbon dioxide being dissolved in the aqueous medium.

According to the invention, the carbon dioxide partial pressure p(CO₂) is less than 0.75 bar absolute and a diluent gas constitutes the remainder of the total pressure.

In an embodiment, a sweep of a diluent gas is maintained in the vapor space above the aqueous medium and carbon dioxide is bubbled through the aqueous medium. The diluent gas is passed into the head space of the reaction vessel without its passing through the aqueous medium so as to maintain a chosen carbon dioxide partial pressure p(CO₂) in the vapor space. It is also possible to deliberately use carbon dioxide as well as a diluent gas to form the atmosphere under which the aqueous medium is held.

Carbon dioxide is bubbled through the aqueous medium, e.g. by means of a nozzle provided at the bottom of the reaction vessel or a gas lance. Excess carbon dioxide comes out of the aqueous medium so as to establish an equilibrium between the dissolved carbon dioxide and the carbon dioxide in the gas phase.

Bubbling carbon dioxide through the aqueous medium is preferred due to faster equilibration and carbon dioxide dissolution. It is also possible, albeit less preferred, to deliberately bubble a mixture of carbon dioxide as well as a diluent gas through the aqueous medium.

In an embodiment, the diluent gas is an inert gas, preferably nitrogen or argon.

The oxidation reaction of tertiary amines yielding amine oxides using hydrogen peroxide is exothermic. For example, the exothermicity of the oxidation reaction of N-methylmorpholine with hydrogen peroxide is about −160 KJ/mol of oxidized N-methylmorpholine. Said exothermic character results in the formation of heat of reaction which mandatorily needs to be removed, e.g., by the use of cooling jackets on the vessel in which the reaction is conducted, in order to control the reaction temperature within a desired range and ensure safe process conditions. To alleviate the problems of heat removal, the aqueous hydrogen peroxide is generally added over a period of time instead of all in a single addition. Dosage time is thus governed by the efficacy of the heat removal which is, among other things, highly impacted by the batch size. Particularly where large batch sizes are attempted, conducting the reaction within an accurate and precise temperature range requires fairly long dosage times of about 2 to 24 hours.

The dosage time is reciprocally correlated with the hydrogen peroxide dosage rate r(H₂O₂). The hydrogen peroxide dosage rate r(H₂O₂) can be expressed as number of moles of hydrogen peroxide being dosed per 1 mol of tertiary amine (based on the stoichiometric amount of tertiary amine initially present) and hour. In an embodiment, the hydrogen peroxide dosage rate r(H₂O₂) is in the range of from 0.035 to less than 1 mol per 1 mol of tertiary amine and hour.

In a further aspect of the invention, it has been surprisingly found that, in order to obtain optimal results, the carbon dioxide partial pressure p(CO₂) and hydrogen peroxide dosage rate (H₂O₂) are preferably coordinated. Hence, in an embodiment, the carbon dioxide partial pressure p(CO₂) in bar absolute is given by

$$p ( {CO}_{2} ) = r ( {H}_{2} {O}_{2} ) / z$$

wherein z is a variable in the range of from 1 to 4 h⁻¹ bar⁻¹, preferably 1.5 to 3 h⁻¹ bar⁻¹.

It is considered that the hydrogen peroxide dosage rate r(H₂O₂) governs the concentration of unreacted hydrogen peroxide available within the aqueous medium, with high dosage rates resulting in higher hydrogen peroxide concentrations before it is consumed in the reaction. Within the limits defined by the equation, it is ensured that a high proportion of carbon dioxide, preferably essentially all of the carbon dioxide, is able to react with hydrogen peroxide forming peroxycarbonate. As a consequence, “free” carbon dioxide in the aqueous medium is avoided or at least substantially reduced.

Conveniently, the hydrogen peroxide is metered at a substantially constant dosage rate. Alternatively, the hydrogen peroxide dosage rate may be varied within the limits of the above equation.

Preferably, the hydrogen peroxide dosage rate is in the range of from 0.035 to 0.5 mol per 1 mol of tertiary amine and hour, more preferably 0.035 to 0.15 mol per 1 mol of tertiary amine and hour.

For example,
(i) when the hydrogen peroxide dosage rate is in the range of 0.5 to 0.25 mol per 1 mol of tertiary amine and hour, the carbon dioxide partial pressure is in range of from 0.06 to 0.5 bar absolute,
(ii) when the hydrogen peroxide dosage rate is in the range of less than 0.25 to 0.1 mol per 1 mol of tertiary amine and hour, the carbon dioxide partial pressure is in the range of from 0.025 to 0.25 bar absolute, and
(iii) when the hydrogen peroxide dosage rate is in the range of less than 0.1 to 0.035 mol per 1 mol of tertiary amine and hour, the carbon dioxide partial pressure is in the range of 0.009 to 0.1 bar absolute.

According to the invention, the total amount of hydrogen peroxide is less than a stoichiometric amount. Preferably, the total amount of hydrogen peroxide used is in the range of from 50 to 99 mol-%, more preferably 80 to less than 90 mol-%, in particular 85 to less than 90 mol-%, per 1 mol of tertiary amine. This ensures essentially complete consumption of the hydrogen peroxide introduced into the reaction. As a consequence, the amine oxide contains less than 50 ppm of hydrogen peroxide, relative to the weight of the amine oxide. Preferably, the amine oxide contains less than 10 ppm of H₂O₂. The concentration of residual hydrogen peroxide can be determined by potentiometric titration with aqueous cerium (IV) sulfate in acidic solution.

In an embodiment, the tertiary amine forms a minimum boiling azeotrope with water at a pressure in the range of from 0.01 to 5 bar absolute. It is sufficient that said azeotrope is formed at one pressure in the mentioned range. Under these circumstances, the unreacted amine may be removed as an azeotrope with water. For example, a suitable tertiary amine is N-methylmorpholine.

In an embodiment, work-up of the reaction mixture comprises subjecting the tertiary amine oxide solution to distillation to remove water and/or water-azeotrope of unreacted tertiary amine. For this purpose, the azeotrope of the unreacted amine with water is distilled off, together with compounds having a boiling point lower than the boiling point of the azeotrope, directly from the crude reaction mixture, preferably in a distillation tower.

Preferably, the distillation is carried out as a continuous distillation. The compounds having a boiling point lower than the boiling point of the azeotrope are withdrawn at the top of the distillation tower, while the azeotrope is withdrawn via a first side-draw of the distillation tower. In an embodiment, the distillation is carried out at a pressure in the range of from 50 to 300 mbar.

The removed water-azeotrope of unreacted tertiary amine is suitably recycled to the reaction with hydrogen peroxide. In the event that the water-azeotrope of unreacted tertiary amine as obtained by the distillation has a concentration of tertiary amine outside the range of from 40 to 95 vol.-%, the concentration of the tertiary amine may be adjusted. This means that either water is added if the concentration of tertiary amine is too high, or tertiary amine is added if the concentration of tertiary amine is too low.

Generally, the starting material of the process, i.e. the tertiary amine, may contain unavoidable impurities. For example, N-methylmorpholine almost invariably contains trace amounts of morpholine. Morpholine may be oxidized in a side-reaction yielding morpholine oxide as a colored side-product. Such colored side-products are critical for the color of the amine oxide product and should therefore be avoided. In order to avoid accumulation of these impurities, the impurities may be suitably purged via an additional side-draw of the continuous distillation tower. The location of the additional side-draw depends on the nature, in particular the boiling point, of the impurity. Mostly, said additional side-draw is located below the first side-draw of the distillation tower.

After distilling off substantially all of the unreacted amine, the remaining residue essentially consists of the amine oxide and water. Amine oxides are commonly stored and/or distributed as aqueous solutions. Advantageously and preferably, the resulting residue essentially consisting of the amine oxide and water is a ready-to-use commercial product which does not require any additional purification or processing steps. However, if necessary, e.g. in the event that a very high amount of water needs to be distilled off in order to remove all of the unreacted amine, additional water may be added to the distillation bottom in order to adjust the concentration of the aqueous amine oxide in view of consumer needs and/or storage stability.

The reaction can be conducted over a wide temperature range. The temperature should be high enough to cause the reaction to proceed at a reasonable rate but not so high as to lead to decomposition of the reactants or products. In an embodiment, the process is conducted at a temperature of 20 to 90° C., preferably 20 to 65° C.

The process of the invention yields a substantially nitrosamine-free amine oxide. In the context of the content of nitrosamine, the term “substantially free” means that nitrosamine(s) is/are present in the amine oxide as a contaminant in a trace amount of less than 50 ppb, relative to the weight of amine oxide.

The process of the invention yields a substantially colorless amine oxide. In the context of the discoloration of the amine oxide, the term “substantially colorless” refers to a 50 wt.-% aqueous solution of the amine oxide having an extinction of less than 2%/cm at 450 nm. The extinction is determined using a UV/VIS spectrometer device (e.g. available from Perkin Elmer) at a temperature of 20° C.

Alternatively, the discoloration of the amine oxide may be characterized via the APHA color number. Preferably, the amine oxide has an APHA color number of less than 100. The APHA color number is a standard method based on a visual comparison of the sample with solutions with known concentrations of cobalt chloroplatinate. The unit of color is that produced by 1 mg platinum/L in the form of the chloroplatinate ion.

The process of the invention allows for obtaining amine oxides which are substantially nitrosamine-free, substantially peroxide-free, and substantially colorless. This unique combination of desirable features has not been achieved so far in a process only comprising oxidation and distillation. Therefore, the invention further relates to a N-methylmorpholine N-oxide having, relative to the weight of N-methylmorpholine N-oxide, a nitrosamine content of less than 50 ppb and a peroxide content of less than 20 ppm, and an extinction of less than 2%/cm at 450 nm as a 50 wt.-% aqueous solution of the N-methylmorpholine N-oxide.

### EXAMPLES

The present invention can be further explained and illustrated on the basis of the following examples.

### Example 1

An aqueous solution of N-methylmorpholine (NMM, 63 wt.-%; 1.01 kg, 1.0 eq.) was placed in a 1.6 L double-walled glass reactor. Said reactor was equipped with a crossbar stirrer, a reflux condenser, a thermometer, a frit-fitted glass tube connected to a CO₂ (3.0) line, a gas inlet (w/o tube) connected to a N₂ (technical) line and a H₂O₂ dosage line (Teflon tube connected to a diaphragm metering pump). H₂O₂ was fed from a glass reservoir. The reaction was carried out at 1.0 bar absolute.

The aqueous solution was heated to a temperature of 50° C. under a constant stream of nitrogen (1.5 L/h). After the temperature of 50° C. was reached, dosage of CO₂ was started at a stream of 0.15 L/h. From the ratio of the streams and the total pressure, a CO₂ partial pressure of 91 mbar can be calculated.

After 60 min, dosage of H₂O₂ was started (50 wt.-% aqueous H₂O₂, 395 g, 0.9 eq.) at a stream of H₂O₂ of 0.93 mL/min (i.e. 0.15 mol/(mol·h); z=1.65 h⁻¹ bar⁻¹). During dosage of H₂O₂, the temperature was maintained at 50° C. and dosage of CO₂ and N₂ was continued.

After the dosage of H₂O₂ was finished, the concentration of H₂O₂ in the reaction mixture was determined by cerimetric titration. Stirring at 50° C. and dosage of CO₂ and N₂ was maintained until the concentration of residual H₂O₂ in the reaction mixture was less than 50 ppm (about 1 h).

Subsequently, dosage of CO₂ was stopped, and the reaction mixture was allowed to cool to room temperature. The reflux condenser was exchanged by a distillation apparatus (condenser and vacuum pump) and water and NMM were distilled off at a sump temperature of 50° C. (2 fractions, first fraction, collected at about 150 mbar: 133 g, second fraction, collected at about 80 mbar: 188 g).

Water (308 g) was added to the distillation residue giving 1328 g of a 49.6 wt.-% aqueous solution of N-methylmorpholine N-oxide (NMMO) having an APHA color number of 22, an extinction at 450 nm of 1.2%, and a N-nitroso morpholine content of less than 20 ppb.

### Example 2

An aqueous solution of N-methylmorpholine (NMM, 63 wt.-%; 1.01 kg, 1.0 eq.) was placed in a 1.6 L double-walled glass reactor. Said reactor was equipped with a crossbar stirrer, a reflux condenser, a thermometer, a frit-fitted glass tube connected to a CO₂ (3.0) line, a gas inlet (w/o tube) connected to a N₂ (technical) line and a H₂O₂ dosage line (Teflon tube connected to a diaphragm metering pump). H₂O₂ was fed from a glass reservoir. The reaction was carried out at 1.0 bar absolute.

The aqueous solution was heated to a temperature of 50° C. under a constant stream of nitrogen (1.5 L/h). After the temperature of 50° C. was reached, dosage of CO₂ was started at a stream of 0.50 L/h. From the ratio of the streams and the total pressure, a CO₂ partial pressure of 250 mbar can be calculated.

After 60 min, dosage of H₂O₂ was started (50 wt.-% aqueous H₂O₂, 395 g, 0.9 eq.) at a stream of H₂O₂ of 0.93 mL/min (i.e. 0.15 mol/(mol·h); z=0.6 h⁻¹ bar1). During dosage of H₂O₂, the temperature was maintained at 50° C. and dosage of CO₂ and N₂ was continued.

After the dosage of H₂O₂ was finished, the concentration of H₂O₂ in the reaction mixture was determined by cerimetric titration. Stirring at 50° C. and dosage of CO₂ and N₂ was maintained until the concentration of residual H₂O₂ in the reaction mixture was less than 50 ppm (about 30 min).

Subsequently, dosage of CO₂ was stopped, and the reaction mixture was allowed to cool to room temperature. The reflux condenser was exchanged by a distillation apparatus (condenser and vacuum pump) and water and NMM were distilled off at a sump temperature of 50° C. (2 fractions, first fraction, collected at about 150 mbar: 145 g, second fraction, collected at about 80 mbar: 162 g).

Water (312 g) was added to the distillation residue giving 1318 g of a 50.1 wt.-% aqueous solution of N-methylmorpholine N-oxide (NMMO) having an APHA color number of 296, an extinction at 450 nm of 4.3%, and a N-nitroso morpholine content of less than 20 ppb.

### Example 3

An aqueous solution of N-methylmorpholine (NMM, 63 wt.-%; 1.01 kg, 1.0 eq.) was placed in a 1.6 L double-walled glass reactor. Said reactor was equipped with a crossbar stirrer, a reflux condenser, a thermometer, a frit-fitted glass tube connected to a CO₂ (3.0) line, a gas inlet (w/o tube) connected to a N₂ (technical) line and a H₂O₂ dosage line (Teflon tube connected to a diaphragm metering pump). H₂O₂ was fed from a glass reservoir. The reaction was carried out at 1.0 bar absolute.

The aqueous solution was heated to a temperature of 50° C. under a constant stream of nitrogen (1.5 L/h). After the temperature of 50° C. was reached, dosage of CO₂ was started at a stream of 0.05 L/h. From the ratio of the streams and the total pressure, a CO₂ partial pressure of 32 mbar can be calculated.

After 60 min, dosage of H₂O₂ was started (50 wt.-% aqueous H₂O₂, 395 g, 0.9 eq.) at a stream of H₂O₂ of 0.93 mL/min (i.e. 0.15 mol/(mol·h), z=4.69 h⁻¹ bar⁻¹). During dosage of H₂O₂, the temperature was maintained at 50° C. and dosage of CO₂ and N₂ was continued.

6 h after the dosage of H₂O₂ was finished, dosage of CO₂ was stopped, and the reaction mixture was allowed to cool to room temperature. The reflux condenser was exchanged by a distillation apparatus (condenser and vacuum pump) and water and NMM were distilled off at a sump temperature of 50° C. (2 fractions, first fraction, collected at about 150 mbar: 143 g, second fraction, collected at about 80 mbar: 170 g).

Water (322 g) was added to the distillation residue giving 1329 g of a 49.6 wt.-% aqueous solution of N-methylmorpholine N-oxide (NMMO) having an APHA color number of 10, an extinction at 450 nm of 0.5%, a H₂O₂ content of 1200 ppm and a N-nitroso morpholine content of 180 ppb.

### Comparative Example 1

N-Methylmorpholine (NMM, 101.1 g, 1.0 eq.) and water (37.5 mL; yielding a mixture of 73 wt.-% NMM and 27 wt.-% water) were placed in a 250 mL double-walled glass reactor. Said reactor was equipped with a crossbar stirrer, a reflux condenser, a thermometer, a frit-fitted glass tube connected to a CO₂ (3.0) line and a H₂O₂ dosage line (Teflon tube connected to a diaphragm metering pump). The head space above the aqueous phase inside the reactor (including the condenser) was about 500 mL H₂O₂ was fed from a glass reservoir. The reaction was carried out at 1.0 bar absolute.

The aqueous solution was heated to a temperature of 50° C. under a constant stream of CO₂ (1.0 L/h) for 2 hours without stirring. In total, 2 L of CO₂ were fed during that time which corresponds to about 4 times the headspace volume of the reactor and thus was sufficient to essentially replace the previous atmosphere by pure CO₂.

Then, dosage of H₂O₂ was started (50 wt.-% aqueous H₂O₂, 61.2 g, 0.9 eq.) at a stream of H₂O₂ of 0.21 mL/min (i.e. 0.225 mol/(mol·h); z=0.23 h⁻¹ bar⁻¹). During dosage of H₂O₂, the temperature was maintained at 50° C. and dosage of CO₂ was continued at a stream of 0.1 L/h.

After the dosage of H₂O₂ was finished, the concentration of H₂O₂ in the reaction mixture was determined by cerimetric titration. Stirring at 50° C. and dosage of CO₂ were maintained until the concentration of H₂O₂ was below 50 ppm (about 10 min).

Subsequently, dosage of CO₂ was stopped, and the reaction mixture was allowed to cool to room temperature. The reflux condenser was exchanged by a distillation apparatus (condenser and vacuum pump) and water and NMM were distilled off at a sump temperature of 50° C. (2 fractions, first fraction, collected at about 150 mbar: 21.9 g, second fraction, collected at about 80 mbar: 25.7 g).

Water (59.2 g) was added to the distillation residue giving 208.4 g of a 50.1 wt.-% aqueous solution of N-methylmorpholine N-oxide (NMMO) having an APHA color number of 297, an extinction at 450 nm of 4.9%, and a N-nitroso morpholine content of less than 20 ppb.

### Comparative Example 2

N-Methylmorpholine (NMM, 101.1 g, 1.0 eq.) and water (37.5 mL; yielding a mixture of 73 wt.-% NMM and 27 wt.-% water) were placed in a 250 ml double-walled glass reactor. Said reactor was equipped with a crossbar stirrer, a reflux condenser, a thermometer, a frit-fitted glass tube connected to a CO₂ (3.0) line and a H₂O₂ dosage line (Teflon tube connected to a diaphragm metering pump). H₂O₂ was fed from a glass reservoir. The reaction was carried out at 1.0 bar absolute.

The aqueous solution was heated to a temperature of 50° C. under a constant stream of CO₂ (0.082 L/h) and nitrogen (0.353 L/h) for 2 hours with stirring. From the ratio of the streams and the total pressure, a CO₂ partial pressure of 188 mbar can be calculated

Then, dosage of H₂O₂ was started (50 wt.-% aqueous H₂O₂, 71.4 g, 1.05 eq.) at a stream of H₂O₂ of 0.34 mL/min (i.e. 0.362 mol/(mol·h); z=1.92 h⁻¹ bar1). During dosage of H₂O₂, the temperature was maintained at 50° C. and dosage of CO₂ and N₂ was continued.

After the dosage of H₂O₂ was finished, stirring at 50° C. and dosage of CO₂ and N₂ were maintained for 16 hours. Subsequently, dosage of CO₂ was stopped, and the reaction mixture was allowed to cool to room temperature.

24.0 g of water were added yielding 233.3 g of a 50.1 wt.-% aqueous solution of N-methylmorpholine N-oxide (NMMO) having an APHA color number of 33, an extinction at 450 nm of 0.6%/cm, and a N-nitroso morpholine content of less than 20 ppb. The mixture contained 0.716 wt.-% of hydrogen peroxide.

To an aliquot of this mixture (about 100 g) manganese dioxide (0.5 g) was added which resulted in vigorous gas formation. As gas formation had ceased, the mixture was essentially hydrogen peroxide free (as determined by cerimetric titration) but APHA color number was 236. Upon standing for 4 days at room temperature, APHA color number further increased, eventually exceeding the scale (>1000) and extension was 6.9%/cm. To another aliquot of this mixture (about 100 g) platinum on charcoal (2.0 g, 57 wt.-% water, Pt-content: 5.0 wt.-% of solids) was added which resulted in vigorous gas formation. As gas formation had ceased, the mixture was essentially hydrogen peroxide free (as determined by cerimetric titration) and APHA color number was 78. Upon standing for 4 days at room temperature, APHA color number increased to 527 and extension was 3.9%/cm.

### Example 4

Triethylamine (101.3 g, 1.0 eq.) and water (10.0 mL; giving a mixture of 91 wt.-% NMM and 9 wt.-% water) were placed in a 250 mL double-walled glass reactor. Said reactor was equipped with a crossbar stirrer, a reflux condenser, a thermometer, a frit-fitted glass tube connected to a CO₂ (3.0) line and a H₂O₂ dosage line (Teflon tube connected to a diaphragm metering pump). H₂O₂ was fed from a glass reservoir. The reaction was carried out at 1.0 bar absolute.

The aqueous mixture was heated to a temperature of 50° C. under a constant stream of nitrogen (0.70 L/h). After the temperature of 50° C. was reached, dosage of CO₂ was started at a stream of 0.164 L/h. From the ratio of the streams and the total pressure, a CO₂ partial pressure of 190 mbar can be calculated.

After 60 min, dosage of H₂O₂ was started (50 wt.-% aqueous H₂O₂, 61.2 g, 0.9 eq.) at a stream of H₂O₂ of 0.286 mL/min (i.e. 0.30 mol/(mol·h); z=1.57 h⁻¹ bar1). During dosage of H₂O₂, the temperature was maintained at 50° C. and dosage of CO₂ and N₂ was continued.

After the dosage of H₂O₂ was finished, the concentration of H₂O₂ in the reaction mixture was determined by cerimetric titration. Stirring at 50° C. and dosage of CO₂ were maintained until the concentration of H₂O₂ was below 50 ppm (about 180 min).

Subsequently, dosage of CO₂ was stopped, and the reaction mixture was allowed to cool to room temperature. The reflux condenser was exchanged by a distillation apparatus (condenser and vacuum pump) and water and triethylamine were distilled off at a sump temperature of 50° C. (1 fraction, collected at about 100 mbar: 35.3 g).

Water (47.6 g) was added to the distillation residue giving 210.1 g of a 49.7 wt.-% aqueous solution of triethylamine N-oxide having an APHA color number of 29, an extinction at 450 nm of 0.8%, and a N-nitroso diethylamine content of less than 20 ppb.

1.-17. (canceled)

18. A process for making a substantially nitrosamine-free, substantially peroxide-free, substantially colorless amine oxide solution, said process comprising reacting a tertiary amine capable of forming an amine oxide with a less than stoichiometric amount of aqueous hydrogen peroxide in an aqueous medium while imposing on the aqueous medium a vapor space having a carbon dioxide partial pressure p(CO₂) of less than 0.75 bar absolute, wherein
the aqueous hydrogen peroxide is metered into an aqueous solution of the tertiary amine at a hydrogen peroxide dosage rate r(H₂O₂) in the range of from 0.035 to less than 1 mol per 1 mol of tertiary amine and hour, and
the carbon dioxide partial pressure p(CO₂) in bar absolute is given by
$$ρ ( C {O}_{2} ) = r ( {H}_{2} {O}_{2} ) / z$$
wherein z is a variable in the range of from 1 to 4 h⁻¹ bar⁻¹.

19. The process of claim 18, wherein a sweep of a diluent gas is maintained in the vapor space above the aqueous medium and carbon dioxide is bubbled through the aqueous medium.

20. The process of claim 19, wherein the diluent gas is an inert gas.

21. The process of claim 18, wherein the concentration of tertiary amine in the aqueous medium is in the range of from 40 to 95 vol.-%.

22. The process of claim 18, wherein the process comprises adding 10 to 70 wt.-% aqueous hydrogen peroxide to the aqueous medium.

23. The process of claim 18, wherein the total amount of hydrogen peroxide used is 50 to 99 mol-% per mol of tertiary amine.

24. The process of claim 18, which is conducted at a temperature of 20 to 90° C.

25. The process of claim 18, wherein the tertiary amine forms a minimum boiling azeotrope with water at a pressure in the range of from 0.01 to 5 bar absolute.

26. The process of claim 25, wherein the tertiary amine is N-methylmorpholine.

27. The process of claim 25, comprising subjecting the tertiary amine oxide solution to distillation to remove water and/or water-azeotrope of unreacted tertiary amine.

28. The process of claim 27, wherein the distillation is carried out at a pressure in the range of from 50 to 300 mbar.

29. The process of claim 27, wherein the removed water-azeotrope of unreacted tertiary amine is recycled to the reaction with hydrogen peroxide.

30. The process of claim 18, wherein the amine oxide has a nitrosamine content of less than 50 ppb, relative to the weight of amine oxide.

31. The process of claim 18, wherein a 50 wt.-% aqueous solution of the amine oxide has an extinction of less than 2%/cm at 450 nm.

32. The process of claim 31, wherein a 50 wt.-% aqueous solution of the amine oxide has an APHA color number of less than 100.
